(19) United States
(12) Patent Application Publication
(10) Pub. No.: US 2025/0155454 A1
(43) Pub. Date: May 15, 2025

(54) METHOD OF DETECTING TISSUE DAMAGE

(71) Applicant: MEDICORTEX FINLAND OY, Turku (FI)

(72) Inventors: Lasse VÄLIMAA, Turku (FI); Adrian HAREL, Turku (FI); Oskar HAAVISTO, Turku (FI); Mårten KVIST, Turku (FI)

(21) Appl. No.: 18/840,320

(22) PCT Filed: Dec. 1, 2022
(86) PCT No.: PCT/FI2022/050805

§ 371 Date: Aug. 21, 2024

(30) Foreign Application Priority Data
Feb. 22, 2022 (FI) 20225156

(51) Int. Cl. G01N 33/68

(52) U.S. Cl. CPC G01N 33/6893; G01N 2333/4724; G01N 2800/60

(57) ABSTRACT

The present invention concerns an in vitro method of detecting a tissue damage, provided that the tissue is not a brain tissue, in a subject. The method includes determining level of glycan-based indicator in at least one body fluid obtainable from the subject, wherein increased level of the glycan-based indicator as compared to a normal control level is indicative of tissue damage in the subject. Also use or certain glycan-based indicators as unique indicators of tissue damage are disclosed.

### FIELD

The present disclosure relates to an in vitro method for detecting tissue damage, in particular to methods wherein the detecting is based on determining level of certain glycan-based indicators such as glycoproteins and cleavage products thereof in body fluid of a subject suspected to suffer from tissue damage, wherein the tissue is not brain tissue. The disclosure also relates to use of glycans as indicators of tissue damage.

### BACKGROUND

Internal tissue damage can be challenging to detect in certain situations. The diagnostic procedure of X-ray or CT imaging to detect bone fractures, for example, exposes patients to irradiation, and it is not available everywhere at all times, yet time-sensitive treatments require prompt decisions. X-ray, CT imaging, and MRI imaging can indicate bone fractures and several soft tissue lesions but there are several disadvantages in using these methods, such as cost, effort and the massive instrumentation needed.

Biochemical markers that indicate tissue damage can guide the decision making whether, for example, an X-ray imaging to investigate a possible bone fracture is needed, and that way potentially avoiding harm from unnecessary examinations.

Comorbidities are a common and severe issue in many diseases and conditions. A serious injury or disease leaves the body more vulnerable than usual to other diseases, and the body is prone to suffer from conditions such as cerebrovascular diseases that can lead to neuronal damage. Patients with underlying conditions have an increased risk of suffering certain comorbidities such as stroke. Detailed assessment of comorbidities is challenging and dangerous if the patient is critically ill, which limits the ability to identify the underlying pathophysiology.

The injury mechanisms of various neurological conditions include either direct neurological damage or inflammation or both. Additionally, other disorders such as sepsis or hypoxia etc. can indirectly lead to tissue damage.

Accordingly, there is need for new methods for detecting tissue damage in general, and orthopaedic damages in particular.

### BRIEF DESCRIPTION OF DRAWINGS

FIG. 1 shows lectins with 80% increase in binding of glycans at statistical significance of p≤0.1 in saliva samples. The y-axis shows the increase (fold change) in the average of injury samples compared to the average of the uninjured healthy control samples which is represented by the y-axis value of 1.0.

FIG. 2 shows lectins with increase in binding of glycans in saliva samples. The y-axis shows the increase (fold change) in the average of injury samples compared to the average of the uninjured healthy control samples which is represented by the y-axis value of 1.00. Black bar: TBI; white bar: orthopaedic damage (shown when the ratio is ≥1.00).

FIG. 3 shows lectins with increase in binding of glycans in urine samples. The y-axis shows the increase (fold change) in the average of injury samples compared to the average of the uninjured healthy control samples which is represented by the y-axis value of 1.00. Black bar: TBI; white bar: orthopaedic damage (shown when the ratio is ≥1.00).

### SUMMARY

The present invention is based on the observation that increase of concentration of certain glycan-based indicators in body fluid can be regarded as an indication of tissue damage, wherein the tissue is not brain tissue.

Accordingly, it is an object of the present invention to provide a new in vitro method of detecting tissue damage in a subject, provided that the tissue is not brain tissue, the method comprising the following steps
a) providing at least one body fluid sample obtainable from said subject,
b) determining level of at least one glycan-based indicator in said at least one body fluid sample,
c) comparing the determined level of said at least one glycan-based indicator to a control level, wherein said control level is the level said at least one glycan-based indicator in corresponding body fluid of an uninjured subject and
d) providing the detecting based on said comparing wherein increased level of said at least glycan-based indicator compared said control level is indicative to tissue damage in said subject.

It is also an object of the present invention to provide use of a glycan-based indicator comprising an N-glycan selected from a group consisting of H3N3P1, H4N3P1, H4N4P1, H3N2P1, H4N5F1P1, H4N4F1P1, H4N5F2P1, H4N4S1, H5N4F3P1, H4N5F3P1, H4N5F1S1, H4N5F2S1, H3N5F1, H9N2, H3N4, H8N2, H7N2, H3N4F1, H3N5, H4N5F1, H4N5, H6N2, H3N4F1P1, preferably from H3N4F1P1, H3N4F1, H3N5F1, H3N5, and H9N2, wherein H is hexose, N is N-acetyl hexosamine, F is fucose, S is sialic acid, and P is sulphate or phosphate ester, as an indicator of orthopaedic damage in saliva.

It is still an object of the present invention to provide use of a glycan-based indicator comprising an N-glycan selected from a group consisting of H11N2P1, H4N4S2G1, H5N5F3S1, H5N6S3, H5N6F1S3, H5N8F1S3, H5N4F2S1, H4N5F1S1, H5N5F1S1, H7N4F1S3P1, H5N6F3S2, preferably from H5N6F1S3 and H11N2P1, wherein H is hexose, N is N-acetyl hexosamine, F is fucose, S is sialic acid, G is N-glycolylneuraminic acid, and P is sulphate or phosphate ester, as an indicator of orthopaedic damage in urine.

It is still an object of the present invention to provide use of a glycan-based indicator comprising an N-glycan selected from a group consisting of H5N5F1S2, H4N5, H4N5F1S1, and H5N5S1, wherein H is hexose, N is N-acetyl hexosamine, F is fucose, S is sialic acid as an indicator of orthopaedic damage in plasma.

It is still an object of the present invention to provide use of a kit or a device in the method according to claim 1, the kit comprising at least one lectin, antibody, or combination thereof that selectively binds to a glycan-based indicator comprising a N-glycan, and a control for comparing to a measured value of binding wherein the glycan-based indicator comprises a N-glycan selected of H3N3P1, H4N3P1, H4N4P1, H3N2P1, H4N5F1P1, H4N4F1P1, H4N5F2P1, H4N4S1, H5N4F3P1, H4N5F3P1, H4N5F1S1, H4N5F2S1, H3N5F1, H9N2, H3N4, H8N2, H7N2, H3N4F1, H3N5, H4N5F1, H4N5, H6N2, H11N2P1, H4N4S2G1, H5N5F3S1, H5N6S3, H5N6F1S3, H5N8F1S3, H5N4F2S1, H4N5F1S1, H5N5F1S1, H7N4F1S3P1, H3N4F1P1, H5N6F3S2, H5N5F1S1, and H5N5S1, wherein H is hexose, N is N-acetyl hexosamine, G is N-glycolylneuraminic acid, F is fucose, and P is sulphate or phosphate ester.

It is still an object of the present invention to provide use of a kit or a device in the method according to claim 1, wherein the glycan-based indicator is a lectin binding indicator, said kit or device comprising at least one lectin selected from the group consisting of PPL, UDA, CALSEPA, ECA, CPA, SHA, LBA, SSA, WGA, BC2L-A, MALECTIN, ASA, HHA (HHL, AL), NPA (NPL, DL), LPA, GRFT, GNA (GNL), DSA (DSL), BANLEC, SNA-I and GNA (GNL), and a control for comparing to a measured value of binding.

Further objects of the present invention are described in the accompanying dependent claims.

Exemplifying and non-limiting embodiments of the invention, both as to constructions and to methods of operation, together with additional objects and advantages thereof, are best understood from the following description of specific exemplifying embodiments when read in connection with the accompanying drawings.

The verbs “to comprise” and “to include” are used in this document as open limitations that neither exclude nor require the existence of un-recited features. The features recited in the accompanied depending claims are mutually freely combinable unless otherwise explicitly stated. Furthermore, it is to be understood that the use of “a” or “an”, i.e., a singular form, throughout this document does not exclude a plurality.

### DESCRIPTION

According to one embodiment the present invention concerns an in vitro method of detecting tissue damage, provided that the tissue is not brain tissue, in a subject, the method comprising the following steps
i. determining level of at least one glycan-based indicator in at least one body fluid sample obtainable from the subject,
ii. comparing the determined level to a control level, wherein the control level is the level said at least one glycan-based indicator in corresponding body fluid of an uninjured subject, and
iii. providing the detecting based on the comparing, wherein increased level of the at least one glycan-based indicator compared to control level is indicative of tissue damage in the subject, provided that the tissue is not brain tissue.

Screening with a glycan indicator panel can give comprehensive information on the status of various vital organs and tissues. It will help doctors in targeting their examination to specific tissues or organs and expedite the diagnosis of a condition or injury. For example, if the patient is unconscious, the tissue damage indicator can serve as a means to easily obtain information on the condition of internal tissues.

A glycan-based tissue damage indicator can also be used to monitor the effectiveness of an intervention or treatment of a disease, or healing of a specific tissue.

The indicator can be used for children, adults, and elderly.

This indicator can originate from various cells, tissues, and organs of the body. The injury/damage can target (but not limited to) one or more of the organs and tissues selected from a group consisting of skin, connective tissue, bones, cartilage, pancreas, glands, blood vessels, adipose tissue (fat). Skeletal muscles, brain, lungs, kidney, liver, heart, lymphatic system, extracellular system, extracellular matrix, peripheral nerves, central nervous system, spleen, and bladder. A particular tissue is bone.

According to one embodiment the damage is an orthopaedic damage. As defined herein an orthopaedic damage or trauma refers to an injury caused by some external force, such as a severe fall, a serious accident, or even a violent attack. These injuries occur in the musculoskeletal system including bones, cartilage, joints, ligaments, muscles, and tendons of the body.

A particular orthopaedic damage is a bone fracture.

When the tissue is bone tissue, the method is for detecting orthopaedic damage.

The injury can also target one or more of the following (but not limited to) cell types: squamous (flattened) epithelial cell, epithelial cells, corneocytes, keratinocytes, Langerhans cells, Merkel cells, melanocytes, fibroblasts, macrophages, adipocytes, mast cells, blood cells (red blood cells, white blood cells, thrombocytes), osteocytes, chondrocytes, endothelial cells, pancreatic cells, secretory cells, smooth muscle cells, muscle cells (myocytes), cardiomyocytes, hepatocytes, Schwann cells, satellite glial cells, neurons, and glial cells (oligodendrocytes, astrocytes, ependymal cells, microglia).

The indicator can occur in one or more of the following body fluids: blood (plasma or serum), cerebrospinal fluid (CSF), urine, saliva (including also spit, mucus, sputum, phlegm, nasal discharge), lymph fluid, lymphatic fluid, interstitial fluid, tears, exudate, sweat, extracellular fluid. Particular fluids suitable for the present method are saliva, urine, and plasma, preferably saliva and urine.

A particular injury which can be detected using the present method is an orthopaedic injury. When an orthopaedic injury occurs and leg, hand or pelvis is broken, different types of cells are damaged, for example, osteocytes of the bone, chondrocytes from cartilage, tendons cells, epithelial cells of blood vessels, myo-cells of muscles, fibroblasts, and the skin as well as connective tissue of broken organ.

As defined herein glycan is an oligosaccharide or carbohydrate portion of a glycoconjugate, such as a glycoprotein, glycolipid, or a proteoglycan. The origin of the glycan-based molecule can be cell content (listed below) that is spilled and undergo metabolic degradation, enzymatic cleavage, or chemical breakdown. Such molecules can originate from the immune system like globulins or antibodies, blood proteins like albumin, or macromolecules originating from intracellular fluid.

The glycan-based indicator suitable for the method is present and detectable in body fluid following tissue injury, in particular orthopaedic injury. The glycan-based indicator can be in its intact, native conformation, in which case altered concentration of the glycan-based indicator in body fluid, in comparison to healthy population, indicates injury. Alternatively, it can be an abnormal biodegradation product, in which case mere emergence of the abnormal product in the body fluid may indicate injury. As a consequence of cell damage, specific proteins are released that start to digest the surrounding proteins inexorably. This leads to the formation or elevation of certain glycan structures in bodily fluids that can indicate cell damage.

In certain embodiments, the glycan-based indicator refer to a glycoprotein, i.e., a protein that has an oligosaccharide (glycan) group attached in post-translational modifications to an amino acid side chain. The glycan is attached via a glycosidic bond which forms between hemiacetal or hemiketal group of the saccharide and either hydroxyl, nitrogen, phosphorus, carbon, or sulphur group of the amino acid which are connoted O-, N-, P-, C- or S-glycosylation, respectively. Common glycosylated amino acids are asparagine, threonine, serine, tyrosine, tryptophan, and cysteine. In some embodiments of the invention, glycan-based molecule can also be in the form a glycosylated fatty acid or lipid. For example, glycosphingolipids are present on cell surface membranes and are particularly abundant in the immune system. Glycosphingolipids are a subtype of glycolipids containing an amino alcohol sphingosine. In general, the glycan may be part of other structure (glycoconjugate, being bound with e.g. protein, peptide, or lipid), or it can be a free-floating carbohydrate, or a degradation product of a glycan or glycoconjugate. The original proteins can be intracellular proteins or blood proteins, including immunoglobulins, or CSF-proteins. Biodegradation product is a molecule generated by exposure to degrading enzymes such as proteolytic or glycolytic enzymes, or some molecules are undergoing cleavage by hydrolysis and chemical reactions.

The glycan-based indicator can also be used to monitor the effectiveness of an intervention or treatment or healing of a specific tissue. The healing can be spontaneous like tissue regeneration or induced by chemical treatment or cellular implant.

The subject as defined herein includes humans and animals, more specifically, mammals.

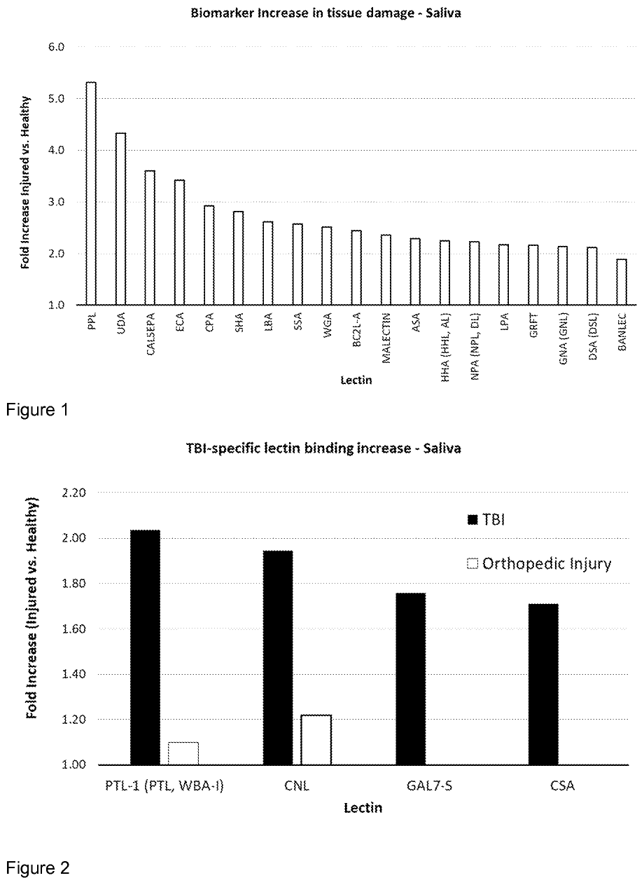

FIG. 1 shows lectins increase in binding of glycans at statistical significance of p≤0.1 in saliva samples. The y-axis shows the increase (fold change) in the average of orthopaedic injury samples compared to the average of the uninjured healthy control samples which is represented by the y-axis value of 1.0. Accordingly, the level of several lectins is increased significantly compared to healthy controls. This is the case with urine also (table 3).

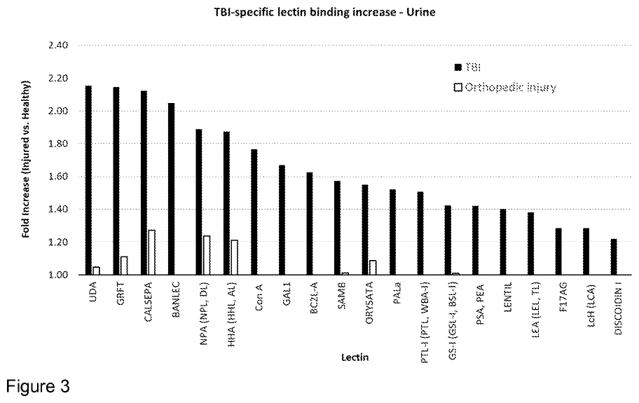

FIGS. 2 and 3 show lectins increase in binding of glycans at statistical significance of p≤0.1 in saliva and urine samples accordingly. As seen from the figures not only an orthopaedic damage but also brain damage gives rise to increase of glycans in body fluids. Accordingly, it is essential to select correct body fluid and glycan for detection.

According to one embodiment the method concerns detecting orthopaedic damage.

According to this embodiment the method comprises the following steps
a) providing saliva, and/or urine sample obtainable from said subject,
b) determining level of binding of at least one glycan-based indicator in said saliva, urine and/or sample to at least one lectin,
c) comparing the determined level of binding to a control level, wherein said control level is level of binding of said at least one glycan-based indicator to said at least one lectin in saliva and/or urine of an uninjured subject and
d) providing the detecting based on said comparing wherein increased level of binding of said at least glycan-based indicator compared to said control level is indicative of orthopaedic damage, in said subject wherein
   for urine, the at least one lectin is selected from a group consisting of: SNA-1 and GNA (GNL),
   for saliva, the at least one lectin is selected from a group consisting of: PPL, UDA, CALSEPA, ECA, CPA, SHA, LBA, SSA, WGA, BC2L-A, MALECTIN, ASA, HHA (HHL, AL), NPA (NPL, DL), LPA, GRFT, GNA (GNL), DSA (DSL), and BANLEC.

According to the method, the increase of binding is preferably 20% compared to the control level.

According to a particular embodiment the method is performed using a lectin array comprising, for urine samples, one of more lectins selected from a group consisting of SNA-1 and GNA (GNL), and for saliva samples, at least one lectin selected from a group consisting of: PPL, UDA, CALSEPA, ECA, CPA, SHA, LBA, SSA, WGA, BC2L-A, MALECTIN, ASA, HHA (HHL, AL), NPA (NPL, DL), LPA, GRFT, GNA (GNL), DSA (DSL), and BANLEC.

The advantage of the embodiments described above is that the structure of the glycan-based indicator does not need to be known. The only requirement is that lectin is specific enough for glycans relevant to the tissue damage in question, such as orthopaedic damage, i.e. the detecting is not distorted by other pathological conditions.

According to another embodiment the method includes analysis of the glycan-based indicators indicative to tissue damage such as orthopaedic damage. The analysis may include treatment of the samples with N-glycosidase and purification through solid-phase extraction. MALDI TOF mass spectrometry is a particular method since it is not prone to fragment the N-glycans.

Accordingly, the in vitro method of detecting orthopaedic damage may comprise also the following steps
a) providing urine, saliva, and/or plasma sample obtainable from said subject,
b) determining level of least one glycan-based indicator in said urine, saliva and/or plasma sample,
c) comparing the determined level to a control level, wherein said control level is level of said at least one glycan-based indicator in saliva, urine and/or plasma of an uninjured subject, and
d) providing the detecting based on said comparing wherein increased level of said at least glycan-based indicator compared said control level is indicative of orthopaedic damage in said subject, wherein
   for urine sample, the at least one glycan-based indicator comprises a N-glycan H11N2P1, H4N4S2G1, H5N5F3S1, H5N6S3, H5N6F1S3, H5N8F1S3, H5N4F2S1, H4N5F1S1, H5N5F1S1, H7N4F1S3P1, H5N6F3S2, preferably from H5N6F1S3 and H11N2P1,
   for saliva sample the at least one glycan-based indicator comprises a N-glycan selected from a group consisting of H3N3P1, H4N3P1, H4N4P1, H3N2P1, H4N5F1P1, H4N4F1P1, H4N5F2P1, H4N4S1, H5N4F3P1, H4N5F3P1, H4N5F1S1, H4N5F2S1, H3N5F1, H9N2, H3N4, H8N2, H7N2, H3N4F1, H3N5, H4N5F1, H4N5, H6N2, H3N4F1P1, preferably from H3N4F1P1, H3N4F1, H3N5F1, H3N5, and H9N2, and
   for plasma sample the at least one glycan-based indicator comprises a N-glycan selected from a group consisting of H5N5F1S2, H4N5, H4N5F1S1, and H5N5S1,
wherein H is hexose, N is N-acetyl hexosamine, F is fucose, S is sialic acid, G is N-glycolylneuraminic acid, and P is sulphate or phosphate ester.

The glycan-based indicator can be either an intact form, or degraded part, of any of the following molecular types: carbohydrate, sugars, glycan, glycolipid, trisaccharide, oligosaccharide, polysaccharide, glycoprotein, glycopeptide, proteoglycan, peptidoglycan, glycopolymer.

The N-glycans can be determined by using one or more of: mass spectrometry, lectin binding, antibody binding, aptamer binding.

The level of N-glycan in the sample can be determined e.g. by one or more of: intensity of the MS signal of the N-glycan, level of lectin binding, level of antibody binding, and level of aptamer binding.

The determining of the glycan-based indicator comprising the N-glycans from body fluid can be done by any method known in the art. Exemplary non-limiting method are mass spectrometry, chromatography and separation methods, affinity binding like lectin binding, antibody binding and aptamer binding, or gel electrophoresis.

When the sample is urine, the lectin binding is preferably determined using an array comprising one or more lectins selected from a group consisting of SNA-I and GNA (GNL).

When the sample is urine, the lectin binding is preferably determined using an array comprising one or more lectins selected from a group consisting of PPL, UDA, CALSEPA, ECA, CPA, SHA, LBA, SSA, WGA, BC2L-A, MALECTIN, ASA, HHA (HHL, AL), NPA (NPL, DL, LPA, GRFT, GNA (GNL), DSA (DSL), and BANLEC.

It was also found that the N-glycan profile in saliva and urine differ significantly from each other. This phenomenon can be considered when selecting the most suitable glycans for tissue damage, such as orthopaedic damage detection.

Preferable glycan structure groups found to be particularly relevant indicators of orthopaedic damage in urine and saliva are collected in Table 1.

TABLE 1
urine

| | |
|---|---|
| 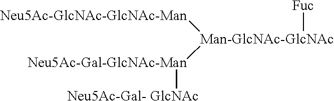 | N-glycans containing terminal N-acetyl hexosamine (N > H and H4N4) For example, H5N6F1S3, where N > H and possibly containing sialylated |
| | LacdiNAc |
| 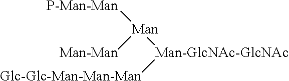 | phosphorylated/ glucosylated high-mannose type glycan H11N2P1 |

saliva

| | |
|---|---|
| 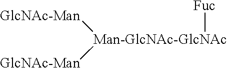 | N-glycans containing terminal N-acetyl hexosamine (N > H) H3N4F1, H3N5F1 H3N5 |
| 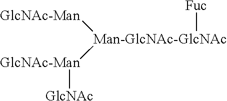 | |
| 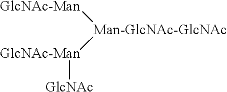 | |
| 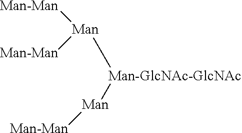 | high-mannose N-glycan H9N2 |
| 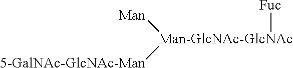 | N-glycans containing terminal N-acetyl hexosamine (N > H and H3N3/H4N4) H3N4F1P1, where N > H and |
| | sulfo-LacdiNAc or |
| 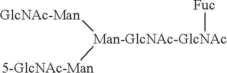 | 6-sulfo-GlcNAc |

Preferable glycan structure groups found to be particularly relevant indicators of orthopaedic damage in plasma are collected in Table 2.

TABLE 2

| plasma | |
|---|---|
| 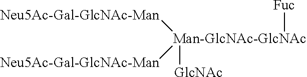 | H5N5F1S2 |
| 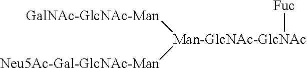 | H4N5F1S1 |
| 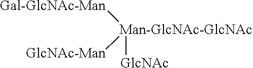 | H4N5 |
| 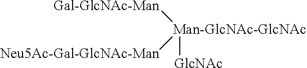 | H5N5S1 |

According to a preferable embodiment the method comprises determining level of plurality of glycan-based indicators, and providing the detecting based on said determined levels of said plurality of glycan-based indicators, wherein increased level of said plurality of glycan-based indicators compared to that of normal control levels is indicative of tissue damage in the subject.

It is also possible to provide plurality of body fluid samples of the subject e.g. a saliva sample, a urine sample, and a plasma sample, determine the level of the at least one glycan-based indicator in the samples, and provide the detecting based on the determined levels, in wherein increased levels of the at least one glycan-based indicator in samples compared to normal control levels is indicative of tissue damage, such as orthopaedic damage in the subject. The use of two or more body fluid samples allows the use of the best indicators in body fluids such as saliva and urine for detecting. According to an exemplary embodiment, glycan-based indicators comprising a N-glycan selected from H5N6F1S3 and H11N2P1 are used as indicators in urine sample, glycan-based indicators comprising a N-glycan selected from H3N4F1, H9N2, H3N4F1P1 are used as indicators in saliva samples and glycan-based indicators comprising a N-glycan selected from H5N5F1S2, H4N5, H4N5F1S1, and H5N5S1 are used as indicators in plasma samples for determining orthopaedic damage.

It is also an object of the present invention to provide a new use of glycan-based indicators as indicator molecules of tissue damage provided that the tissue is not brain tissue.

When the body fluid is saliva, the glycan-based indicators comprising a N-glycan selected from a group consisting of H3N3P1, H4N3P1, H4N4P1, H3N2P1, H4N5F1P1, H4N4F1P1, H4N5F2P1, H4N4S1, H5N4F3P1, H4N5F3P1, H4N5F1S1, H4N5F2S1, H3N5F1, H9N2, H3N4, H8N2, H7N2, H3N4F1, H3N5, H4N5F1, H4N5, H6N2, H3N4F1P1, preferably H3N4F1, H9N2, H3N4F1P1 wherein H is hexose, N is N-acetyl hexosamine, F is fucose, S is sialic acid, and P is sulphate or phosphate ester can be used as indicators of orthopaedic damage. Preferable N-glycan structures suitable for indicators of orthopaedic damage in saliva are shown in table 1.

When the body fluid is urine, the glycan-based indicators comprising a N-glycan selected from a group consisting of H11N2P1, H4N4S2G1, H5N5F3S1, H5N6S3, H5N6F1S3, H5N8F1S3, H5N4F2S1, H4N5F1S1, H5N5F1S1, H7N4F1S3P1, H5N6F3S2, preferably H5N6F1S3 and H11N2P1 wherein H is hexose, N is N-acetyl hexosamine, F is fucose, S is sialic acid, G is N-glycolylneuraminic acid, and P is sulphate or phosphate ester can be used as indicators of orthopaedic damage. Preferable N-glycan structures suitable for indicators of orthopaedic damage in urine samples are shown in table 1.

When the body fluid is plasma the glycan-based indicators comprising a N-glycan selected from a group consisting of H5N5F1S2, H4N5, H4N5F1S1, and H5N5S1 wherein H is hexose, N is N-acetyl hexosamine, F is fucose, S is sialic acid, can be used as indicators of orthopaedic damage.

It is still an object of the present invention to provide a kit or a device for use in the method. The kit or the device comprises at least one lectin, antibody, or combination thereof that selectively binds to a glycan-based indicator comprising a N-glycan, and a control for comparing to a measured value of binding. The N-glycan of the glycan based indicator of the kit or the device is selected from a group consisting of H3N3P1, H4N3P1, H4N4P1, H3N2P1, H4N5F1P1, H4N4F1P1, H4N5F2P1, H4N4S1, H5N4F3P1, H4N5F3P1, H4N5F1S1, H4N5F2S1, H3N5F1, H9N2, H3N4, H8N2, H7N2, H3N4F1, H3N5, H4N5F1, H4N5, H6N2, H11N2P1, H4N4S2G1, H5N5F3S1, H5N6S3, H5N6F1S3, H5N8F1S3, H5N4F2S1, H4N5F1S1, H5N5F1S1, H7N4F1S3P1, H3N4F1P1, and H5N6F3S2, wherein H is hexose, N is N-acetyl hexosamine, F is fucose, and S is sialic acid, G is N-glycolylneuraminic acid, and P is sulphate or phosphate ester. The kit can be uses for determine of orthopaedic damage. The lectin of the kit is preferably selected from a group consisting of PPL, UDA, CALSEPA, ECA, CPA, SHA, LBA, SSA, WGA, BC2L-A, MALECTIN, ASA, HHA (HHL, AL), NPA (NPL, DL), LPA, GRFT, GNA (GNL), DSA (DSL), BANLEC, SNA-I and GNA (GNL).

It is still an object of the present invention to provide use a kit or a device in the method of the invention comprising at least one lectin selected from a group consisting of PPL, UDA, CALSEPA, ECA, CPA, SHA, LBA, SSA, WGA, BC2L-A, MALECTIN, ASA, HHA (HHL, AL), NPA (NPL, DL), LPA, GRFT, GNA (GNL), DSA (DSL), BANLEC, SNA-I and GNA (GNL).

The kit can also comprise a washing solution or instructions for making a washing solution, in which the combination of the capture reagents and the washing solution allows capture of the indicators on the solid support or column for subsequent detection by, e.g., antibodies or mass spectrometry. In a further embodiment, a kit can comprise instructions for suitable operational parameters in the form of a label or separate insert. For example, the instructions may inform a healthcare professional or a consumer about how to collect the sample, how to wash the probe or the particular indicators to be detected, etc. In yet another embodiment, the kit can comprise one or more containers with indicator samples, to be used as standard(s) for calibration.

As is apparent to a skilled person, the present lectin array kit can be used with either a label-based method or as a sandwich-based method. In one embodiment, the label-based method is used for biotinylated samples containing proteoglycans and glycoproteins or glycopeptides for direct detection on the array via a Cy3 equivalent dye-conjugated Biotin-Streptavidin complex. In another embodiment, a sandwich-based method is used for antibody detection of glycocalyx elements (glycolipids, glycoproteins, etc.) captured on the array. Labelled reporter antibodies specific for the glycocalyx elements of interest may be provided in the kit or supplied by the user of the kit. An example protocol for this procedure with a general “Antibody Cocktail” may be included in a user manual. In some non-limiting embodiments, specific antibody concentrations and conditions may need to be determined by the end user.

In one embodiment of the indicator detection kit, HRP protein or other enzymes and fluorescent light or change in absorbance may be employed in order to detect the indicator in a body fluid and to indicate the quantity of the indicator in percentage. This may be incorporated into a portable application that indicates the severity of orthopaedic damage on a scale comprising, but not limited to, none, mild, moderate, and severe. In another embodiment, an analogous yes/no reply is received. These examples do not exclude other possible embodiments.

In some embodiments, the present invention provides use of at least one antibody in a kit or in a device to detect tissue damage, such as orthopaedic damage, where the antibody may be a polyclonal or a monoclonal antibody of any species, or a fragment thereof, either enzymatically cleaved or recombinantly produced, or a humanized antibody, and where the antibody recognizes and binds glycan, glycoprotein, peptidoglycan, proteoglycan, glycolipid, protein, small molecule, lectin, or antibody of another species (generally ‘antigens’). Said antibody may be used, for instance, as
i) a capture reagent, wherein the antibody is immobilized on a solid substrate to bind its antigen from a sample medium;
ii) an antibody that is immobilized on a solid substrate to bind an analyte-specific capture reagent (for example lectin) so that the bound agent (lectin) is able to capture the analyte (glycan) from a sample;
iii) a primary detection reagent, wherein an antibody conjugated to any label (labelled antibody) recognizes and directly binds an antigen;
iv) a secondary detection reagent, wherein a labelled antibody recognizes and binds a primary detection reagent that is bound to the analyte. For example, a labelled antibody binds to a lectin that has bound to its cognate glycan, or a labelled antibody from one species (e.g. goat) that recognizes and binds an antibody of another species (e.g. mouse) which has bound its antigen;
v) an antibody for recognizing and binding a non-glycan part of a glycan-containing molecule, e.g. a glycoprotein, where the glycoprotein or a fragment thereof is first bound to e.g. lectin via its glycan moiety and then is recognized and bound by an antibody that is specific to the peptide part of the molecule; or
vi) antibody for use in immunoblotting assays.

The kit may also comprise a combination of antibodies for different purposes as mentioned above.

All embodiments, details, advantages, and the like of the present kit also apply to a device for use in different aspects and embodiments of the present invention. Also, all embodiments, details, advantages, and the like of the present methods apply to the present kit, and vice versa. In particular, one or more compounds, compositions, or reagents disclosed as suitable for carrying out the present methods may be comprised in the present kit. Likewise, anything disclosed with reference to the kit, apply to the present methods as well.

The glycan-based indicator can be detected from a body fluid using methods known in the art. Exemplary methods include bio-affinity/ligand binding, mass spectrometry, ligand binding based on complementary structures, and immunochromatographic tests, such as a lateral flow test. Bio-affinity binding assays can be based on, inter alia, glycan-binding lectins, antibodies, or they can utilize synthetic molecular imprinted surfaces (MIP). The recognition and binding of the glycan-based biomolecule can be based on one-sided reaction wherein the target glycan is recognized and bound by a single type of the binder. It can also be a two-sided binding wherein the target glycan is recognized and bound by two different types of the binder each reacting with a separate part of the target glycan-based biomolecule. The option of dual binding of a lectin and antibody targeting the same indicator can increase the specificity as well as improve signal in the detection.

The glycan-based molecules that indicate orthopaedic damage can guide the decision making whether, for example, an X-ray imaging to investigate a possible bone fracture is needed, and if not needed, that way potentially avoiding harm from unnecessary examinations. A clear indication for the need of testing are cases in which the injured patient cannot communicate the location and the severity of the injury. It can be due to language barrier, the patient being drunk or under the influence of drugs, or in many cases, the patient fainted or lost consciousness. The test can be performed even without interaction with the suspected injured patient.

### Experimental

Saliva, urine, and plasma were collected from healthy controls and patients with an orthopaedic injury. Three Finnish hospitals enrolled the patients and collected the samples reaching total numbers of 16 orthopaedic patients and 29 uninjured controls.

The elevation of glycans following injury were observed with a lectin array. The samples were centrifuged and dialyzed, followed by the chemical conjugation of biotin and incubation on the lectin array. The glycans and glycoconjugates bound on the lectin array via their carbohydrate structures were detected with fluorescent label conjugated to streptavidin.

The screening method is semi-quantitative, the fluorescent signal measured from each lectin spot on the array is proportional to the amount of respective lectin-binding-glycan in the sample. These readings can be compared between the injured and healthy groups in order to count a relative difference in the amount of the glycan structures between injured patients and uninjured healthy subjects. The protocol was customized for each body fluid. The clinical samples were analysed on a 95-lectin array. Table 2 and 3 summarise relevant lectins and their glycan-binding specificities and the response in urine and saliva, respectively following an orthopaedic injury.

In addition to the lectin, the samples were analysed with mass spectrometry (MS) to study the carbohydrate structure of the indicator.

Molecules released from the damaged cells may undergo degradation by e.g. hydrolysis, enzymatic cleavage, or chemical breakdown. Glycoproteins can be degraded to glycopeptides and the glycan residues can be removed/liberated completely from the core or the parent structure (protein, peptide, fatty acid, etc.) and appear as free glycans and oligosaccharides. The degradation can be a normal homeostatic process, or it can be unusual because the damaged cells release enzymes and compounds that can digest the glycans uncontrollably leading to unique glycan fingerprints. Damaged bones and bone related tissue, on the other hand, was found to induce the elevation of other specific glycan structures in urine and saliva samples.

TABLE 3
Lectins with ≥20% increase in binding of glycans at
statistical significance of p ≤ 0.1 in urine samples.
The average of the injury samples (Ortho) is compared
to the average of uninjured healthy control (HC) samples.

| Lectin | Full name or origin | Ortho/HC |
|---|---|---|
| SNA-I | Sambucus nigra I | 1.30 |
| GNA (GNL) | Galanthus nivalis | 1.29 |

TABLE 4
Lectins with ≥80% increase in binding of glycans at
statistical significance of p ≤ 0.1 in saliva samples.
The average of the injury samples (Ortho) is compared
to the average of uninjured healthy control (HC) samples.

| Lectin | Full name or origin | Ortho/HC |
|---|---|---|
| PPL | Pleurocybella porrigens lectin | 5.32 |
| UDA | Urtica dioica | 4.33 |
| CALSEPA | Calystegia sepium lectin | 3.60 |
| ECA | Erythrina cristagalli | 3.42 |
| CPA | Cicer arietinum lectin | 2.92 |
| SHA | Salivia horminum lectin | 2.81 |
| LBA | Phaseolus lunatus | 2.61 |
| SSA | Salvia sclarea lectin | 2.57 |
| WGA | Triticum vulgaris (wheat germ) | 2.51 |
| BC2L-A | Burkholderia cenocepacia lectin | 2.44 |
| MALECTIN | Human malectin lectin | 2.36 |
| ASA | Allium sativum | 2.28 |
| HHA (HHL, AL) | Hippeastrum hybrid | 2.24 |
| NPA (NPL, DL) | Narcissus pseudo narcissus | 2.22 |
| LPA | Limulus polyphemus | 2.17 |
| GRFT | Griffithia sp. Lectin | 2.15 |
| GNA (GNL) | Galanthus nivalis | 2.13 |
| DSA (DSL) | Datura stramonium | 2.12 |
| BANLEC | Musa acuminata lectin | 1.89 |

### Urine

Some trend was seen that leg injuries showed higher response compared to arm and small bones injuries. This may be either explained by the fact that lesions in legs are greater than in arm and they release higher amounts of markers, or that the glycans are injury location specific.

### Saliva

The saliva samples were centrifuged and dialyzed, followed by the chemical conjugation of biotin, incubation on the lectin array, and detection by fluorescent-labeled streptavidin. A number of glycan-based molecules were elevated in saliva. The detecting was performed using lectin arrays.

In the healthy samples, glycans such as H3N1, H3N1F2, H4N1 and H5N1 are relatively more abundant compared to the samples from taken injured patients. The structures of these glycans are unknown, but they comprise more hexose residues than the saccharides associated with the injury samples. The series of glycans H1N1, H2N1, H3N1, H4N1 and H5N1 resembles intracellular digestion products that are often seen in total tissue and cellular glycomes (Suzuki & Funakoshi (2006) Free N-linked oligosaccharide chains: Formation and degradation. Glycoconjugate Journal, 23: 291-302.)

1. An in vitro method of detecting a tissue damage in a subject, provided that the tissue is not brain tissue, the method comprising the following steps
a) providing at least one body fluid sample obtainable from said subject,
b) determining level of at least one glycan-based indicator in said at least one body fluid sample,
c) comparing the determined level of said at least one glycan-based indicator to a control level, wherein said control level is level of said at least one glycan-based indicator in corresponding body fluid of an uninjured subject, and
d) providing the detecting based on said comparing, wherein increased level of said at least glycan-based indicator compared said control levels is indicative of tissue damage in said subject.

2. The method according to claim 1 wherein the at least one body fluid is selected from a group consisting of blood, plasma, serum, cerebrospinal fluid, urine, saliva, lymph fluid, lymphatic fluid, interstitial fluid, tears, exudate, sweat, extracellular fluid, preferably saliva, urine, and plasma, more preferably saliva and urine.

3. The method according to claim 1 wherein the tissue is selected from a group consisting of skin tissue, connective tissue, bone, cartilage, pancreas, glands, blood vessels, adipose tissue, skeletal muscle tissue, lung tissue, kidney tissue, liver tissue, heart tissue, lymphatic system tissue, extracellular system tissue, extracellular matrix tissue, peripheral nerve tissue, central nervous system tissue, spleen tissue, and bladder tissue, preferably bone tissue.

4. The method according claim 1, wherein the at least one body fluid is saliva and/or urine, and wherein
step b) comprises determining level of binding of said at least one glycan-based indicator in said urine and/or saliva sample to at least one lectin,
step c) comprises comparing the determined level of binding to a control level, wherein said control level is level of binding of said at least one glycan-based indicator to said at least one lectin in saliva and/or urine of an uninjured subject and
step d) comprises providing the detecting based on said comparing, wherein increased level of binding of said at least glycan-based indicator compared said control levels is indicative of orthopaedic damage in said subject, wherein
for urine, the at least one lectin is selected from a group consisting of: SNA-I and GNA (GNL),
for saliva, the at least one lectin is selected from a group consisting of: PPL, UDA, CALSEPA, ECA, CPA, SHA, LBA, SSA, WGA, BC2L-A, MALECTIN, ASA, HHA (HHL, AL), NPA (NPL, DL), LPA, GRFT, GNA (GNL), DSA (DSL), and BANLEC and wherein increased level of binding of said at least glycan-based indicator compared said control levels is indicative of orthopaedic damage in said subject.

5. The method according to claim 1 wherein the at least one body fluid comprises saliva, urine and/or plasma, and wherein
for urine, the at least one glycan-based indicator comprises a N-glycan selected from a group consisting of H11N2P1, H4N4S2G1, H5N5F3S1, H5N6S3, H5N6F1S3, H5N8F1S3, H5N4F2S1, H4N5F1S1, H5N5F1S1, H7N4F1S3P1, H5N6F3S2,
for saliva, the at least one glycan-based indicator comprises a N-glycan selected from a group consisting of H3N3P1, H4N3P1, H4N4P1, H3N2P1, H4N5F1P1, H4N4F1P1, H4N5F2P1, H4N4S1, H5N4F3P1, H4N5F3P1, H4N5F1S1, H4N5F2S1, H3N5F1, H9N2, H3N4, H8N2, H7N2, H3N4F1, H3N5, H4N5F1, H4N5, H6N2, H3N4F1P1,
for plasma, the N-glycan is selected from a group consisting of H5N5F1S2, H4N5, H4N5F1S1, and H5N5S1, wherein H is hexose, N is N-acetyl hexosamine, F is fucose, S is sialic acid, G is N-glycolylneuraminic acid, and P is sulphate or phosphate ester,
wherein H is hexose, N is N-acetyl hexosamine, F is fucose, S is sialic acid, G is N-glycolylneuraminic acid, and P is sulphate or phosphate ester, and wherein increased level of said at least one glycan-based indicator compared to said control level is indicative of orthopaedic damage in said subject.

6. The method according to claim 5 wherein for the urine sample the N-glycan is selected from H5N6F1S3 and H11N2P1.

7. The method according to claim 5, wherein for saliva sample the N-glycan is selected from a group consisting of H3N4F1, H3N5F1, H3N5, H3N4F1P1 and H9N2.

8. The method according to claim 5 wherein

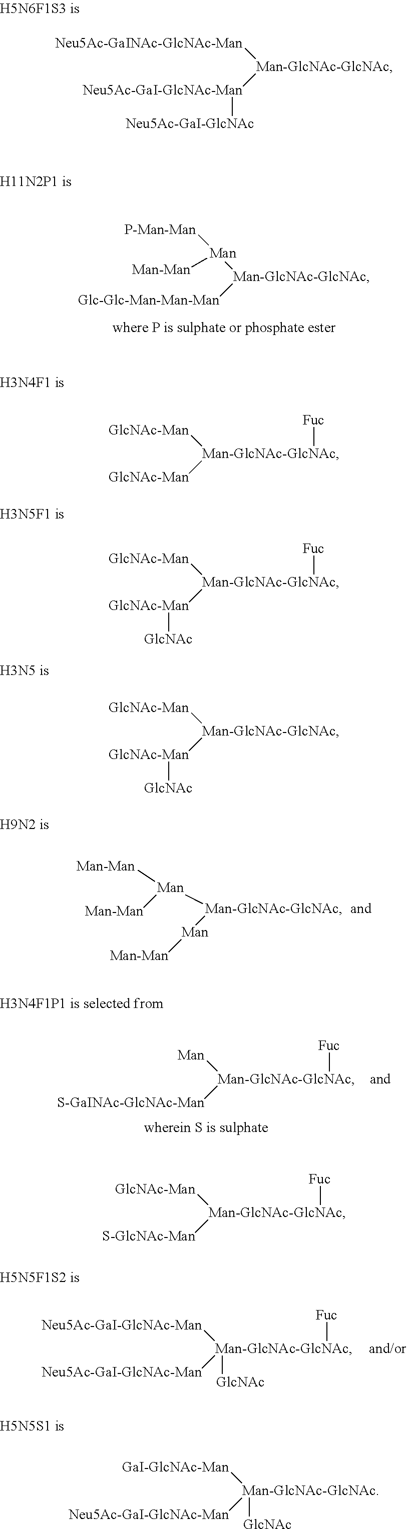

9. The method according to claim 1 wherein the glycan-based indicator is a glycoprotein or a cleavage product thereof.

10. The method according to claim 1 wherein the determining of step b) comprises
i) separating the at least one glycan-based indicator from the sample,
ii) treating the separated glycan-based indicator with N-glycosidase to provide the N-glycan, and
iii) determining level of the N-glycan of the glycan-based indicator by mass spectrometry.

11. The method according to claim 1 wherein the level of the at least one glycan-based indicator in said at least one body fluid sample and said control level is determined by using one or more of mass spectrometry, lectin binding, antibody binding, aptamer binding.

12. The method according to claim 11 wherein the sample is urine, and the lectin binding is determined by using an array comprising one or more lectins selected from a group consisting of SNA-I and GNA (GNL).

13. The method according to claim 11 wherein the sample is saliva, and the lectin binding is determined by using an array comprising one or more lectins selected from a group consisting of: PPL, UDA, CALSEPA, ECA, CPA, SHA, LBA, SSA, WGA, BC2L-A, MALECTIN, ASA, HHA (HHL, AL), NPA (NPL, DL), LPA, GRFT, GNA (GNL), DSA (DSL), and BANLEC.

14-19. (canceled)
